(19) United States
(12) Patent Application Publication
(10) Pub. No.: US 2023/0218019 A1
(43) Pub. Date: Jul. 13, 2023

(54) MEDICAL MASKS, AND METHODS OF MAKING AND USING SAME

(71) Applicants: The United States Govemment as represensted by The Department of Veterans Affairs, Washington, DC (US); REGENTS OF THE UNIVERSITY OF CALIFORNIA, Oakland, CA (US)

(72) Inventor: Scott C. Fuller, Sacramento, CA (US)

(21) Appl. No.: 17/927,193

(22) PCT Filed: May 21, 2021
(86) PCT No.: PCT/US2021/033605

§ 371 Date: Nov. 22, 2022

Related U.S. Application Data
(60) Provisional application No. 63/029,052, filed on May 22, 2020.

(51) Int. Cl. A41D 13/11; A61B 90/00

(52) U.S. Cl. CPC A41D 13/11; A61B 90/05; A61M 2210/0618

(57) ABSTRACT

A mask can comprise a covering material that is sufficiently sized to extend over a mouth and a nose on a head of a wearer. The covering material can have an outer surface and an inner surface. The covering material can define an opening therethrough. At least one transparent membrane can be coupled to the covering material and cover the opening in the covering material. The at least one transparent membrane can define a hole therethrough. An elongate conduit can define an interior passage having a central axis. The elongate conduit can be coupled to the at least one transparent membrane so that the central axis of the interior passage is aligned with the hole. The mask can further comprise at least one securing element for securing the mask to the head of the wearer.

### CROSS-REFERENCE TO RELATED APPLICATION

This application claims priority to and the benefit of the filing date of U.S. Provisional Patent Application No. 63/029,052, filed May 22, 2020, which application is incorporated herein by reference in its entirety.

### FIELD

This application is directed to devices and methods for performing procedures on patients while limiting spread of infection and reducing intervals between uses of examination rooms.

### BACKGROUND

Preventing communication of infection between a patient and medical professionals is a known concern in medicine. COVID-19 has introduced additional challenges, for example to otolaryngologists. Data from Iran, China, and Italy demonstrated increased rates of infection in this discipline that are predicted to be proximate to the intimate nature of the head and neck examination, viral load in the nasopharynx, as well as the tools and techniques associated with examining and treating diseases of the nose and throat, including flexible/rigid fiber optic endoscopy, which are currently characterized as aerosol generating procedures (AGP). As such, performance of a comprehensive head and neck examination on COVID-19 positive and indeterminate patients necessitates use of appropriate PPE (N95 respirator, face shield or goggles, gown, and gloves) to minimize the risk to the providers and cross contamination. Furthermore, aerosol generating procedures done in the standard outpatient clinical setting with commercial air exchange levels require quarantine of examination rooms up to 3 hours before they can be terminally cleaned for further use. This presents a significant impediment to efficient patient throughput at a single facility given the backlog of both new and existing patients who require this form of examination either for surveillance, treatment, or diagnosis, let alone hundreds of thousands of facilities world-wide. This problem of throughput has both enormous financial and clinical consequences if patients cannot be seen in a timely manner.

### SUMMARY

Disclosed herein, in some aspects, is a mask having a covering material, at least one transparent membrane, a membrane reinforcement structure, and at least one securing element. The covering material can be sufficiently sized to extend over a mouth and a nose on a head of a wearer. The covering material can have an outer surface and an inner surface. The covering material can define an opening therethrough. The at least one transparent membrane can be coupled to the covering material and cover the opening in the covering material. The at least one transparent membrane can define a hole therethrough. The membrane reinforcement structure can surround the hole through the at least one transparent membrane. The at least one securing element can be configured to secure the mask to the head of the wearer.

Optionally, the mask can further comprise a valve that is configured to inhibit flow through the hole defined by the at least one transparent membrane. Optionally, the valve can comprise a flexible polymer membrane and a slit through the flexible polymer membrane.

Optionally, the membrane reinforcement structure can comprise an elongate conduit defining an interior passage having a central axis. The elongate conduit can be coupled to the at least one transparent membrane so that the central axis of the interior passage is aligned with the hole. The elongate conduit can define a radially extending flange having an inner flange surface and an outer flange surface. Optionally, the at least one transparent membrane can comprise an inner layer and an outer layer. The inner layer can couple to the inner flange surface, and the outer layer can couple to the outer flange surface. Optionally, the outer layer can couple to the outer surface of the covering material, and the inner layer can couple to the inner surface of the covering material.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a schematic of an inner surface of a mask in accordance with embodiments disclosed herein.

FIG. 2 is a schematic of a side view of the mask as in FIG. 1.

FIG. 3 is a schematic of a portion of a side view of the mask of FIG. 1.

FIG. 4 is a schematic of a cross section of the mask as in FIG. 1.

FIG. shows an outer surface of a mask in accordance with embodiments disclosed herein.

FIG. 6 is a side view of the mask of FIG. 5.

FIG. 7 depicts an inner surface of the mask of FIG. 5.

### DETAILED DESCRIPTION

The present invention can be understood more readily by reference to the following detailed description and appendix, which include examples, drawings, and claims. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a membrane” can include two or more such membranes unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the term “proximal” refers to a direction toward a patient, while the term “distal” refers to a direction away from a patient.

As used herein, the terms “optional” or “optionally” mean that the subsequently described event or circumstance may or may not occur, and that the description includes instances where said event or circumstance occurs and instances where it does not.

Surgical masks can reduce generated aerosols for certain operations. However, mask protection degrades with simple perforation of the mask to allow for passage of endoscopes. Surgical masks can be modified to allow an endoscope alone to be inserted. However, no modified surgical masks have provisions for the passage of additional instruments. Furthermore, visualization is poor with such modified masks, making use of said modified surgical masks challenging to the user and posing a risk to the patient via blind passage of instrumentation. Still further, introduction and manipulation of instruments can result in shearing of such modified masks, thereby opening up a large aperture though which aerosol can be transmitted.

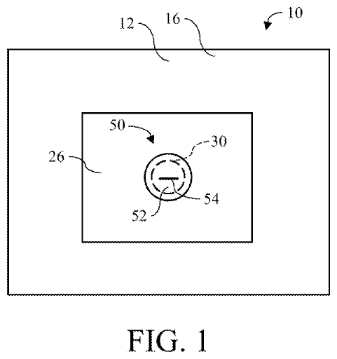

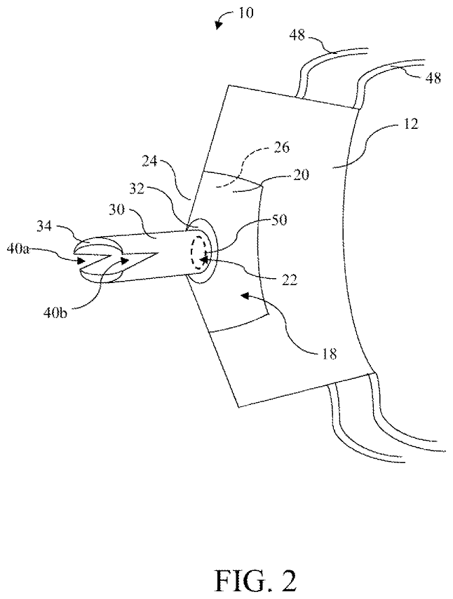

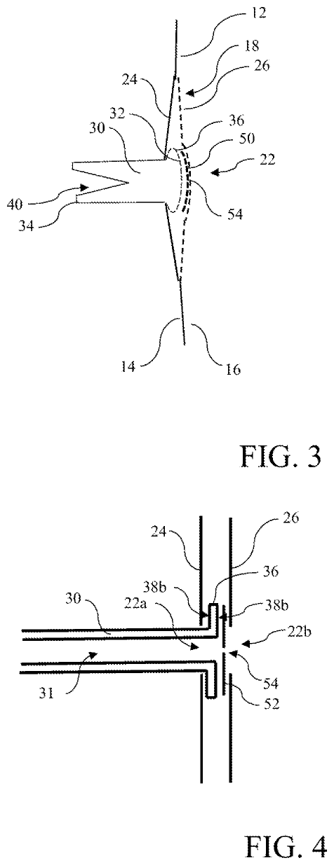

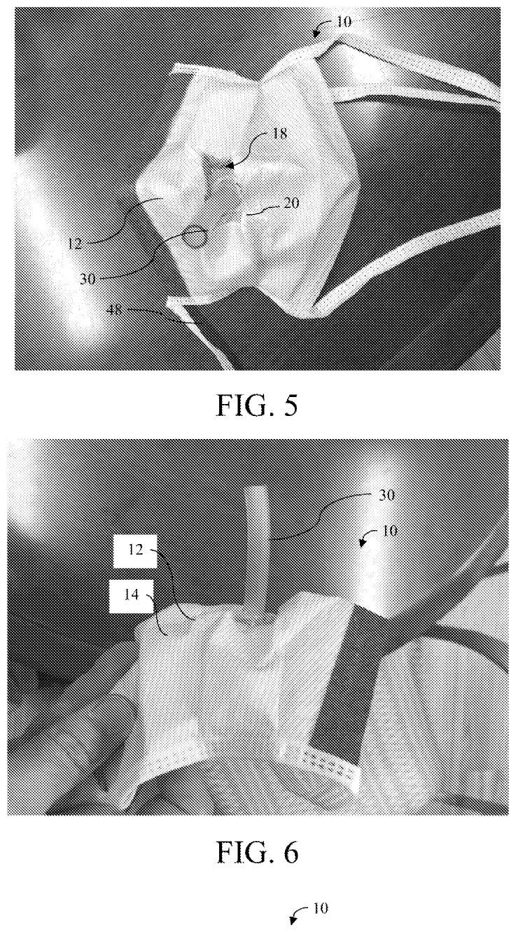

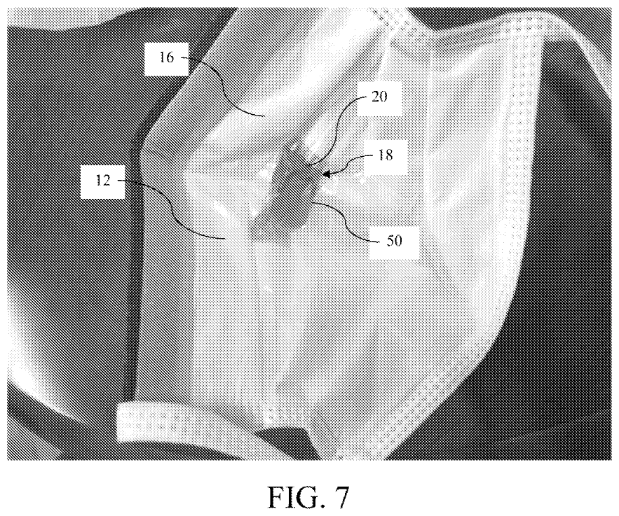

Disclosed herein, with reference to FIGS. 1-7, is a medical mask 10 that can be used during aerosol generating procedures of the head and neck to reduce or eliminate droplet and aerosol distribution of contaminants by a patient. Such aerosol generating procedures include, for example and without limitation, endoscopy, positive pressure ventilation (BiPAP and CPAP), endotracheal intubation, airway suction, high frequency oscillatory ventilation, tracheostomy, chest physiotherapy, nebulizer treatment, sputum induction, and bronchoscopy. However, it is contemplated that the disclosed mask can be used during performance of any examination or treatment of any disease or condition of the head and/or throat. The mask 10 can comprise a covering material 12. The covering material 12 can be sufficiently sized to extend over a mouth and a nose of a wearer. The covering material can have an outer surface 14 and an inner surface 16. The covering material can define an opening 18 therethrough. The opening 18 can optionally be about three centimeters wide (extending relative to top and bottom portions of the face (vertically)) and five centimeters long (extending relative to left and right sides of the face (horizontally)), or about four centimeters wide and about five centimeters long. However, openings of other sizes are contemplated depending upon the particular size, anatomy, and/or conditions of the patient. The opening can optionally be centered or generally centered (e.g., within 15%, within 10%, or within 5% of being centered) with respect to the length and width of the covering material. It is contemplated that the opening can be positioned over the nose and mouth of the wearer.

In some aspects, the covering material 12 can be embodied as a surgical mask with an opening therethrough. The covering material 12 can comprise a nonwoven material as is common in surgical masks. More particularly, it is contemplated that the nonwoven material can comprise polypropylene. Optionally, the non-woven material can comprise multiple layers, such as, for example, three layers. For example, in these aspects, the non-woven material can comprise a melt-blown polymer (e.g., polypropylene) placed between two layers of non-woven fabric, with the melt-blown material configured to filter microbes from entering or exiting the mask. Optionally, the covering material can comprise pleats, which can assist with expansion of the mask in a conventional manner.

In some optional aspects, the covering material can have dimensions of at least 12 cm by at least 18 cm (optionally, at least 12 cm by at least 20 cm). However, it is contemplated that the covering material (and the mask) can have any desired dimensions based upon particular characteristics of a patient, including size, anatomy, and/or condition.

At least one transparent membrane 20 can be coupled to the covering material and extend across the opening 18 in the covering material 12. The transparent membrane(s) 20 can sealingly couple to the covering material 12 so that the air is generally inhibited from passing between outer edges 22 of the transparent membrane and the covering material. In some optional aspects, the transparent membrane(s) 20 can be adhesively secured to the covering material. Additionally, or alternatively, in other optional aspects, it is contemplated that the transparent membrane(s) 20 can be laminated to the covering material 12. The opening 18 and transparent membrane 20 can cooperatively provide a viewing window that enables a medical professional (e.g., a clinician or physician) to see the mouth and/or nose of the wearer for orienting instruments. As disclosed herein, “transparent” should be understood to include various degrees of transparency from fully transparent, in which all or substantially all visible light passes through undistorted, to partially transparent, in which some light is absorbed or distorted while still allowing the medical professional to generally discern the facial features of the wearer. For example, it is contemplated that the transparent membrane(s) can be sufficiently transparent to see locations of the nasal vestibules of the patient. In some optional aspects, the membrane(s) can have an opacity of from about 5% to about 8%, or from 0% to 20%. In some optional aspects, the membrane(s) 20 can comprise polyethylene terephthalate glycol-modified (PETG). In some optional aspects, the at least one transparent membrane 20 can comprise an outer membrane 24 (positioned farthest from the face) and an inner membrane 26 (positioned closest to the face). Optionally, the outer membrane 24 can couple (e.g., adhesively couple) to the outer surface 14 of the covering material 12, and the inner membrane 26 can couple (e.g., adhesively couple) to the inner surface 16 of the covering material 12. Although two membranes 20 are shown in the illustrated embodiments, it is contemplated that the at least one membrane 20 can have one, three, four, or more membrane layers. In some aspects, the membrane(s) 20 can have a textured (e.g., rough) side and a smooth side. It is contemplated that inner-most (or, optionally, only) membrane can be oriented so that the textured side opposes the face of the wearer, as the textured side can inhibit or resist fogging.

The at least one transparent membrane 20 can define a hole 22 therethrough. For example, in embodiments comprising an outer membrane 24 and an inner membrane 26, the outer membrane 24 and the inner membrane 26 can define respective holes 22a, 22b that are axially aligned (optionally, concentrically aligned) to cooperatively form the hole 22. An elongate conduit 30, defining an interior passage 31, can be axially aligned with the center of the hole 22. In this way, the hole 22 and the elongate conduit 30 can cooperate to enable passage of medical instruments through the mask 10. For example, the elongate conduit can define an inner diameter that is sufficient to receive an endoscope therethrough. It is further contemplated that the elongate conduit dimensions can be selected based on a particular procedure and the size of the instrumentation used therewith. For example, in some aspects, the conduit can define an inner diameter of about 1.5 cm or more than 1.5 cm (e.g., between 1.5 cm and 5 cm). In this way, additional instruments (additional to an endoscope) can be passed through the conduit 30. The hole 22 can be positioned so that the proximal end (i.e., the end closest to the patient) of the conduit 30 can be selectively positioned at either nasal vestibule of the nose or the mouth of the wearer.

The elongate conduit 30 can have a proximal end 32 and a distal end 34 that is spaced from the proximal end 32 by a length. In some optional aspects, the elongate conduit 30 can define a radially extending flange 36 at the proximal end. The flange can have an outer flange surface 38a and an inner flange surface 38b. The flange 36 can facilitate coupling between the elongate conduit 30 and the transparent membrane(s). In some optional aspects, the outer membrane 24 can couple (e.g., adhesively couple) to the outer flange surface, and the inner membrane 26 can couple (e.g., adhesively couple) to the inner flange surface 38b. In some optional aspects, the outer and inner membranes 24, 26 can comprise TEGADERM film dressings (3M Medical) or other transparent film dressings. Optionally, the elongate conduit 30 can be embodied as, or comprise features of, at least a portion of a nasal airway tool, such as, for example, a nasopharyngeal airway (or nasal trumpet or nose hose) as is known in the art. It is contemplated that the coupling between the flange 36 of the conduit 30 and the transparent membranes can provide sufficient integrity to enable the elongate conduit to act as a toggle for manipulating the position of the medical instruments with respect to the wearer's face without shearing the mask (thereby inhibiting communication of aerosolized particles therethrough). In some optional aspects, the elongate conduit can comprise one or more polymer materials. In these aspects, the elongate conduit can comprise a flexible material, such as rubber (e.g., a synthetic rubber such as NEOPRENE (DUPONT)).

In some aspects, the elongate conduit 30 can comprise notches 40. The notches 40 can extend from the distal end 34 of the elongate conduit 30. The notches 40 can comprise at least a pair of notches 40a, 40b that are positioned on opposing transverse sides of the conduit. The notches 40 can optionally be positioned on the left and right sides of the elongate conduit 30 (relative to the wearer when the mask is properly worn). The notches 40 can enable greater degrees of lateral freedom when using instrumentation than when the notches are omitted. In some optional aspects, the notches 40 can be tapered in a direction away from the distal end 34. In further optional aspects, the notches 40 can define cuts or separations between opposing sides of the conduit. In this way, the opposing sides of the conduit at the distal ends can be spread to enhance articulation of instrument(s) inserted through the conduit 30, while still enabling the conduit to be used as a toggle for manipulating the instrument(s) inserted therethrough. It is contemplated that the notches 40 can extend from the distal end 34 of the conduit 30 to the flange 36. It is contemplated that the notches can optionally terminate at the flange (i.e., not extend through the flange), maintaining the flange intact so that the flange can provide structural integrity to the mask and inhibit shearing/tearing of the mask. Thus, it is contemplated that the elongate conduit 30 can define a membrane reinforcement structure that is configured to reinforce the hole through the membrane(s) 20. In still further optional aspects, it is contemplated that the elongate conduit 30 can be omitted from the mask 10, and an alternative membrane reinforcement structure can be included to reinforce the hole 22 through the membrane(s) 20. For example, a polymer or metal ring can be coupled to the membrane(s) 20 surrounding the hole 22 to provide structural integrity to inhibit tearing of the membrane(s). It is contemplated that the membrane reinforcement structure can further be sized and shaped (e.g., a tube having an axial length of at least one-half centimeter) to enable a medical professional to manipulate the membrane reinforcement structure for positioning of the hole 22 and instruments inserted therethrough. In various aspects, the elongate conduit 30 (or other reinforcement structure) can comprise acrylate. In further aspects, the elongate conduit 30 (or other reinforcement structure) can comprise nylon, polypropylene, or other suitable material.

At least one securing element 48 can be configured to secure the mask 10 to the wearer. In some aspects, the securing elements 48 can be a pair of elastic straps that attach to opposing sides of the covering material and define loops that are configured to extend around the ears of the wearer. In further aspects, the securing element can be one or more straps that define loops that are configured to extend around the back of the head of the wearer. It is contemplated that other securing elements, such as adhesives, can be used to secure the mask to the face of the wearer. Optionally, a combination of multiple types of securing elements can be provided.

In some aspects, the mask 10 can comprise a valve 50 that is configured to control flow through the elongate conduit (e.g., inhibit flow therethrough when no instrument is inserted through the valve). The valve can comprise a membrane 52 (e.g., a latex or nitrile membrane) that extends across the hole in the at least one transparent membrane. The membrane can define a slit 54 that can open as instruments are inserted therethrough. When no instrument is inserted through the valve, the valve can remain closed to inhibit flow therethrough. When an instrument is passed through the membrane, the membrane can conform to the instruments, thereby inhibiting flow around the instrument (e.g., between the membrane and the instrument). In this way, the valve can minimize escape of aerosolized particles, thereby minimizing risk to healthcare providers (e.g., during aerosol generating procedures). Optionally, the valve 50 can have a thickness of from about 0.002 inches to about 0.015 inches (e.g., about 0.005 inches).

In some aspects, the valve 50 can substantially cover (e.g., with at least 80% of surface area covered) or entirely cover the inner flange surface 38b of the flange 36. The inner membrane 26 can couple (e.g., adhesively couple) to the outer membrane 24 surrounding the flange 36 of the conduit 30 and the valve, thereby securing the valve 50 against the conduit 30 and fixing the position of the proximal end 32 of the conduit 30 with respect to the inner and outer membranes 24, 26.

In various aspects, one or more adhesives can couple together components described herein. For example, the covering material 12 can couple to the membrane(s) 20 via adhesive. In further aspects, ultrasonic welding can be used to couple together one or more components described herein.

In use, by minimizing the escape of aerosolized particles, the mask 10 can reduce delay in room utilization via quarantine to accommodate the necessary room air changes per hour (ACH) to clear aerosolized particles. For example, conventional quarantine times without use of the mask as disclosed herein can range from 15 minutes (OR grade HVAC 20-25 ACH) to 3 hours (commercial office HVAC of 2-3 ACH). In some aspects, the mask can allow for complex examination of the upper aerodigestive tract. The mask can reduce the use of personal protective equipment (PPE) for various procedures (e.g., routine endoscopic examinations) as well as the potential to greatly improve clinic efficiency by eliminating aerosolization of particles and the attendant need to quarantine examination rooms for extended periods of time with resultant decrease in clinical productivity. The mask can increase physician and patient safety and improve patient throughput and diagnosis, thereby diminishing morbidity from waiting to process patient backlog from diseases such as COVID-19.

When using the mask 10, the elongate conduit 30 can serve as a toggle that can enable a medical professional to guide an instrument (e.g., an endoscope) to a desired location (e.g., into a nostril of the wearer of the mask). For example, the mask can be placed over the mouth and nose of the wearer. The medical professional can manipulate the elongate conduit 30 to adjust the position of the proximal end 32 of the elongate conduit 30 as well as the angle of orientation of the elongate conduit. The medical professional can insert an endoscope through the conduit 30 and into the mouth or nose of the patient to perform an endoscopy. In further aspects, the mask 10 can be used with procedures such as bronchoscopies and upper gastro-intestinal endoscopies.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A mask comprising: a covering material that is sufficiently sized to extend over a mouth and a nose on a head of a wearer, wherein the covering material has an outer surface and an inner surface, wherein the covering material defines an opening therethrough; at least one transparent membrane coupled to the covering material and covering the opening in the covering material, wherein the at least one transparent membrane defines a hole therethrough; a membrane reinforcement structure surrounding the hole through the at least one transparent membrane; and at least one securing element for securing the mask to the head of the wearer.

Aspect 2: The mask of aspect 1, further comprising a valve that is configured to inhibit flow through the hole through the at least one transparent membrane.

Aspect 3: The mask of aspect 2, wherein the valve comprises a flexible polymer membrane and a slit through the flexible polymer membrane.

Aspect 4: The mask of any one of the preceding aspects, wherein the membrane reinforcement structure comprises an elongate conduit defining an interior passage having a central axis, wherein the elongate conduit is coupled to the at least one transparent membrane so that the central axis of the interior passage is aligned with the hole, wherein the elongate conduit defines a radially extending flange having an inner flange surface and an outer flange surface.

Aspect 5: The mask of aspect 4, wherein the at least one transparent membrane comprises an inner layer and an outer layer, wherein the inner layer couples to the inner flange surface, and wherein the outer layer couples to the outer flange surface.

Aspect 6: The mask of aspect 5, wherein the outer layer couples to the outer surface of the covering material, and wherein the inner layer couples to the inner surface of the covering material.

Aspect 7: The mask of any one of aspects 4-6, wherein the elongate conduit comprises a pair of notches that extend longitudinally from a distal end of the elongate conduit and are positioned on opposing transverse sides of the elongate conduit.

Aspect 8: The mask of any one of aspects 4-7, wherein the length of the elongate conduit is at least two centimeters.

Aspect 9: The mask of any one of the preceding aspects, wherein the covering material comprises a nonwoven fabric.

Aspect 10: The mask of any one of the preceding aspects, wherein the at least one securing element comprises at least one strap.

Aspect 11: The mask of aspect 9, wherein the at least one strap comprises a first strap that is configured to go around a first ear of the wearer and a second strap that is configured to go around a second ear of the wearer.

Aspect 12: The mask of any one of aspects 4-11, wherein the elongate conduit comprises a nasal airway tool.

Aspect 13: The mask of any one of the preceding aspects, wherein the at least one transparent membrane comprises Tegaderm or PETG.

Aspect 14: The mask of any one of aspects 4-13, wherein the hole through at least one transparent membrane is positioned so that the proximal end of the elongate conduit can be positioned at either nasal vestibule of the nose of the wearer.

Aspect 15: The mask of aspect 14, wherein the hole through at least one transparent membrane is generally centered on the covering material relative to a length dimension and a width dimension.

Aspect 16: The mask of any one of the preceding aspects, wherein the opening through the covering material has dimensions of at least three centimeters by at least five centimeters.

Aspect 17: The mask of any one of the preceding aspects, wherein the covering material has dimensions of at least twenty centimeters by at least twelve centimeters.

Aspect 18: A method of using a mask as in any one of the preceding aspects for a medical procedure, the method comprising: positioning the mask on a patient; and inserting a medical instrument through the membrane reinforcement structure.

Aspect 19: The method of aspect 18, wherein the medical procedure is an endoscopy procedure, and wherein the medical instrument is an endoscope.

Aspect 20: The method of aspect 18 or aspect 19, further comprising using the elongate conduit to orient the endoscope prior to insertion of the endoscope into the patient.

Aspect 21: A method of making a mask as in any one of aspects 1-17.

Although several embodiments of the invention have been disclosed in the foregoing specification and the following appendices, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

1. A mask comprising:
a covering material that is sufficiently sized to extend over a mouth and a nose on a head of a wearer, wherein the covering material has an outer surface and an inner surface, wherein the covering material defines an opening therethrough;
at least one transparent membrane coupled to the covering material and covering the opening in the covering material, wherein the at least one transparent membrane defines a hole therethrough;
a membrane reinforcement structure surrounding the hole through the at least one transparent membrane; and
at least one securing element for securing the mask to the head of the wearer.

2. The mask of claim 1, further comprising a valve that is configured to inhibit flow through the hole through the at least one transparent membrane.

3. The mask of claim 2, wherein the valve comprises a flexible polymer membrane and a slit through the flexible polymer membrane.

4. The mask of claim 1, wherein the membrane reinforcement structure comprises an elongate conduit defining an interior passage having a central axis, wherein the elongate conduit is coupled to the at least one transparent membrane so that the central axis of the interior passage is aligned with the hole, wherein the elongate conduit defines a radially extending flange having an inner flange surface and an outer flange surface.

5. The mask of claim 4, wherein the at least one transparent membrane comprises an inner layer and an outer layer, wherein the inner layer couples to the inner flange surface, and wherein the outer layer couples to the outer flange surface.

6. The mask of claim 5, wherein the outer layer couples to the outer surface of the covering material, and wherein the inner layer couples to the inner surface of the covering material.

7. The mask of claim 4, wherein the elongate conduit comprises a pair of notches that extend longitudinally from a distal end of the elongate conduit and are positioned on opposing transverse sides of the elongate conduit.

8. The mask of claim 4, wherein the length of the elongate conduit is at least two centimeters.

9. The mask of claim 4, wherein the covering material comprises a nonwoven fabric.

10. The mask of claim 4, wherein the at least one securing element comprises at least one strap.

11. The mask of claim 9, wherein the at least one strap comprises a first strap that is configured to go around a first ear of the wearer and a second strap that is configured to go around a second ear of the wearer.

12. The mask of claim 4, wherein the elongate conduit comprises a nasal airway tool.

13. The mask of claim 4, wherein the at least one transparent membrane comprises PETG.

14. The mask of claim 4, wherein the hole through at least one transparent membrane is positioned so that the proximal end of the elongate conduit can be positioned at either nasal vestibule of the nose of the wearer.

15. The mask of claim 14, wherein the hole through at least one transparent membrane is generally centered on the covering material relative to a length dimension and a width dimension.

16. The mask of claim 1, wherein the opening through the covering material has dimensions of at least three centimeters by at least five centimeters.

17. The mask of claim 1, wherein the covering material has dimensions of at least twenty centimeters by at least twelve centimeters.

18. A method of using a mask as in claim 1 for a medical procedure, the method comprising:
positioning the mask on a patient; and
inserting a medical instrument through the membrane reinforcement structure.

19. The method of claim 18, wherein the medical procedure is an endoscopy procedure, and wherein the medical instrument is an endoscope.

20. The method of claim 18, wherein the membrane reinforcement structure comprises an elongate conduit defining an interior passage having a central axis, the method further comprising using the elongate conduit to orient the endoscope prior to insertion of the endoscope into the patient.
